SEQUENCE LISTING (2 sequences)

SEQ ID NO: 1 - protein, 22 aa, synthetic construct
  REGION 1..22: note = LEN Peptide
DIVMTQSPDSLAVSLGERATIN

SEQ ID NO: 2 - protein, 34 aa, synthetic construct
  REGION 1..34: note = Control Peptide
AVSEHQLLHDKGKSIQDLRRRFFLHHLIAEIHTA

(19) United States
(12) Patent Application Publication
(10) Pub. No.: US 2025/0298033 A1
(43) Pub. Date: Sep. 25, 2025

(54) HIGH SENSITIVITY BIOTINYLATED PEPTIDE BINDING ELISA ASSAY

(71) Applicant: Alexion Pharmaceuticals, Inc., Boston, MA (US)

(72) Inventors: Daria Sizova, Bethany, CT (US); Melissa Batonick, Old Saybrook, CT (US)

(73) Assignee: Alexion Pharmaceuticals, Inc., Boston, MA (US)

(21) Appl. No.: 18/862,120

(22) PCT Filed: May 11, 2023
(86) PCT No.: PCT/US2023/021826

§ 371 Date: Oct. 31, 2024

Related U.S. Application Data
(60) Provisional application No. 63/340,766, filed on May 11, 2022.

(51) Int. Cl. G01N 33/68; G01N 33/543; G01N 33/58

(52) U.S. Cl. CPC G01N 33/6854; G01N 33/54366; G01N 33/581

(57) ABSTRACT

Immunoglobulin light Chain “AL” amyloidosis is the most common form of systemic amyloidosis, accounting for approximately 70% of the diagnosed cases in developed countries. As there are treatments available, e.g., based on antibody C11-1F4, and in development for AL amyloidosis, there is a need in for more accurate methods for quality control of c11-1F4 antibodies or antigen-binding fragment thereof. Immunoassays, methods, and kits for testing the specificity of 11-1F4 antibodies are provided. The immunoassays, methods, and kits are more than 10 times more sensitive than the ELISA protocol that is routinely used for evaluation of c11-1F4 binding to LEN peptide.

### CROSS-REFERENCE TO RELATED APPLICATIONS

This application claims the benefit of priority of U.S. Provisional Patent Application No. 63/340,766, filed May 11, 2022, which is incorporated herein by reference in its entirety.

### REFERENCE TO SEQUENCE LISTING SUBMITTED ELECTRONICALLY

The instant application contains a Sequence Listing which has been submitted electronically in XML format, and the entire contents of the Sequence Listing are incorporated herein by reference. Said XML copy, created on May 1, 2023, is named 0657_WO_SL.xml and is 3,360 bytes in size.

### BACKGROUND

Immunoglobulin light Chain (AL) amyloidosis is the most common form of systemic amyloidosis, accounting for approximately 70% of the diagnosed cases in developed countries. As there are treatments available, e.g., based on antibody C11-1F4, and in development for AL amyloidosis, there is a need in for more accurate methods for quality control of c11-1F4 antibodies or antigen-binding fragment thereof.

### SUMMARY

In an embodiment, an immunoassay system can comprise a solid substrate coated with a biotinylated LEN peptide comprising the amino acid sequence of SEQ ID NO: 1 and a biotinylated control peptide comprising the amino acid sequence of SEQ ID NO:2.

In an embodiment, the LEN peptide can consist of the amino acid sequence of SEQ ID NO: 1.

In an embodiment, the control peptide can consist of the amino acid sequence of SEQ ID NO: 2.

In an embodiment, the solid substrate can be coated with the biotinylated LEN peptide at a concentration of between about 0.1 μg/mL and 1 μg/mL. The solid substrate can be coated with the biotinylated LEN peptide at a concentration of about 0.1, 0.15, 0.2, 0.25, 0.3, 0.35, 0.4, 0.45, 0.5, 0.55, 0.65, 0.60, 0.70. 0.75, 0.80, 0.85, 0.90, 0.95, or 1.0 1 μg/mL. The solid substrate can be coated with the biotinylated LEN peptide at a concentration of about 0.1 μg/mL.

In an embodiment, the solid substrate can be coated with the biotinylated control peptide at a concentration of between about 0.1 μg/mL and 1 μg/mL. The solid substrate can be coated with the biotinylated control peptide at a concentration of about 0.1, 0.15, 0.2. 0.25, 0.3, 0.35, 0.4, 0.45, 0.5. 0.55, 0.65, 0.60, 0.70. 0.75, 0.80, 0.85, 0.90, 0.95, or 1.0 1 μg/mL. The solid substrate can be coated with the control peptide at a concentration of about 0.1 μg/mL

In an embodiment, the solid support can be a bead, plate, matrix, polymer, test tube, sheet, culture dish or test strip. The plate can be a multiwell plate. The multiwall plate can consist of a 2-well plate, 4-well plate, 6-well plate-, 12-well plate, 24-well plate, 48-well plate. 96-well plate, or a 384-well plate.

In an embodiment, the solid substrate can be a polystyrene, polypropylene, cyclo-olefin, or a glass solid substrate.

In an embodiment, a kit can comprise the immunoassay system of any one of claims 1-13 and a secondary antibody that specifically binds the 11-1F4 antibody or antigen-binding fragment thereof. The antibody can be conjugated to a detectable label. The detectable label can be a fluorescent label, luminescent label, bioluminescent label, radioactive label, chemiluminescent label, colorimetric label, fluorogenic label, enzymatic label, or a combination thereof. The label can be horseradish peroxidase (HRP). The 11-1F4 antibody can be a chimeric 11-1F4 antibody (c11-1F4 antibody) or antigen-binding fragment thereof. The 11-1F4 antibody can be a humanized 11-1F4 antibody or antigen-binding fragment thereof.

In an embodiment, a method for testing a 11-1F4 antibody binding specificity, can comprise: contacting a 11-1F4 antibody or antigen-binding fragment thereof with the immunoassay system described herein; washing the solid support; adding a secondary antibody that specifically binds the 11-1F4 antibody or antigen-binding fragment thereof; washing the solid support; and executing detection steps.

In an embodiment, the wash can comprise at least 3 washes with a wash buffer. The wash buffer can comprise PBS and 0.05% TWEEN-20 (polysorbate 20).

In an embodiment, the solid support can be coated with a biotinylated LEN peptide comprising the amino acid sequence of SEQ ID NO: 1. The solid support can be coated with a biotiny lated LEN peptide comprising the amino acid sequence of SEQ ID NO: 1, and comprising incubating at solid support with a solution comprising 0.1 μm/mL biotinylated LEN peptide comprising the amino acid sequence of SEQ ID NO: 1 for about 1 hour at about 37° C. The solution can comprise PBS (Phosphate Buffered Saline).

In an embodiment, the solid support can be coated with a biotinylated control peptide comprising the amino acid sequence of SEQ ID NO:2. The solid support can be coated with a biotinylated control peptide comprising the amino acid sequence of SEQ ID NO:2, and comprising incubating at solid support with a solution comprising 0.1 μm/mL biotinylated control peptide comprising the amino acid sequence of SEQ ID NO:2 for about 1 hour at about 37° C. The solution can comprise PBS (Phosphate Buffered Saline).

In an embodiment, the method can further comprise a blocking step prior to step (a) comprising incubating the plate with blocking buffer comprising PBS and 1% BSA for about 60 minutes at about 37° C.

In an embodiment, the method can further comprise adding a control antibody in the absence of an 11-1F4 antibody to the solid support.

In an embodiment, the control antibody can be a human IgG1-k antibody.

In an embodiment, the secondary antibody that specifically binds the 11-1F4 antibody or antigen-binding fragment thereof can be conjugated to a detectable label.

In an embodiment, the detectable label can be a fluorescent label, luminescent label, bioluminescent label, radioactive label, chemiluminescent label, colorimetric label, fluorogenic label, enzymatic label, or a combination thereof. The label can be horseradish peroxidase (HRP).

In an embodiment, a method for testing a 11-1F4 antibody binding specificity can comprise providing a solid support; coating at least a portion of the solid support with a biotiny lated LEN peptide comprising the amino acid sequence of SEQ ID NO: 1 comprising incubating at solid support with a solution comprising 0.1 μm/mL biotinylated LEN peptide comprising the amino acid sequence of SEQ ID NO: 1 for about 1 hour at about 37° C.; coating at least a portion of the solid support with a biotinylated control comprising the amino acid sequence of SEQ ID NO:2 comprising incubating at solid support with a solution comprising 0.1μm/mL biotinylated control peptide comprising the amino acid sequence of SEQ ID NO:2 for about 1 hour at about 37° C.; washing the solid support, optionally at least three times; contacting the solid support with a blocking buffer comprising PBS (Phosphate Buffer Saline) and 1% BSA (Bovine Serum Albumin) and incubating for about 1 hour at about 37° C.; contacting a solution comprising a 11-1F4 antibody or antigen-binding fragment thereof with the solid support and incubating for about 1 hour at about 37° C.; washing the solid support, optionally at least three times; adding a secondary antibody that specifically binds the 11-1F4 antibody or antigen-binding fragment thereof and incubating for about 1 hour at about 37° C.; washing the solid support, optionally at least three times; and executing detection steps.

In an embodiment, the solution can comprise a 11-1F4 antibody or antigen-binding fragment thereof comprises assay buffer comprising PBS (Phosphate Buffer Saline), 1% BSA (Bovine Serum Albumin), and 0.05% TWEEN-20 (polysorbate 20).

In an embodiment, the secondary antibody can be conjugated to a detectable label. The detectable label can be a fluorescent label, luminescent label, bioluminescent label, radioactive label, chemiluminescent label, colorimetric label, fluorogenic label, enzymatic label, or a combination thereof. The label can be horseradish peroxidase (HRP).

In an embodiment, the solution comprising a 11-1F4 antibody or antigen-binding fragment thereof can comprise a series of serial dilutions of the solution 11-1F4 antibody or antigen-binding fragment thereof.

In an embodiment, the 11-1F4 antibody can be a chimeric 11-1F4 antibody (c11-1F4 antibody) or antigen-binding fragment thereof.

In an embodiment, the 11-1F4 antibody can be a humanized 11-1F4 antibody or antigen-binding fragment thereof.

### BRIEF DESCRIPTION OF THE DRAWINGS

For a further understanding of the nature, objects, and advantages of the present disclosure, reference should be had to the following detailed description, read in conjunction with the following drawings.

FIG. 1 depicts the combined data from three independent assays. EC₅₀=2.6±0.2 ng/ml (0.018±0.001 nM). Biotinylated and control peptide coating concentration: 0.1 μg/ml.

FIG. 2 depicts a schematic map of an 8×12 plate, showing antibody concentration (rows A through H), the antibody used (columns 1 through 6 for c11-1F4 and columns 7 through 9 for IgG1-K), and control (columns 10-12) for an exemplary biotinylated LEN peptide plate (1 μg/mL).

FIG. 3 depicts a schematic map of an 8×12 plate, showing antibody concentration (rows A through H), the antibody used (columns 1 through 6 for c11-1F4 and columns 7 through 9 for IgG1-K), and control (columns 10-12) for an exemplary biotinylated control peptide plate (1 μg/mL).

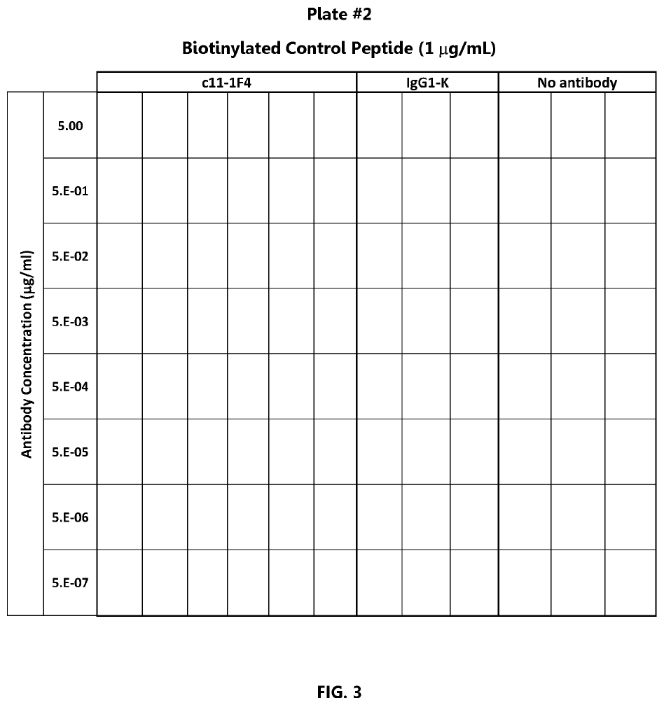

### DETAILED DESCRIPTION

It is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

The singular forms “a,” “an” and “the” include plural reference unless the context clearly dictates otherwise. Unless defined otherwise, all technical and scientific terms used herein have the same meaning as commonly understood to one of ordinary skill in the art to which this disclosure belongs.

### Definitions

“Antibodies” (Abs) and “immunoglobulins” (Igs) are glycoproteins having the same structural characteristics. While antibodies exhibit binding specificity to a specific antigen. immunoglobulins include both antibodies and other antibody-like molecules that lack antigen specificity. The term “antibody” specifically covers monoclonal antibodies, including antibody fragment clones.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light-and heavy-chain variable domains. Chothia et al. J. Mol. Biol. 186:651 (1985); Novotny and Haber Proc. Natl. Acad. Sci. U.S.A. 82:4592 (1985).

“Variable,” as used herein, refers broadly to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. Johnson & Wu “Kabat Database and its applications: 30 years after the first variability plot.” Nucleic Acids Res. (2000) 28(1): 214-218. The constant domains are not involved directly in binding an antibody to an antigen, but exhibit various effector functions, such as participation of the antibody in antibody-dependent cellular toxicity.

Papain digestion of antibodies produces two identical antigen-binding fragments called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen-recognition and binding site. In a two-chain Fv species, this region consists of a dimer of one heavy and one light chain variable domain in tight, non-covalent association. In a single-chain Fv species, one heavy and one light chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgF, IgG, and IgM, and several of these can be further divided into subclasses (isotypes), e.g., IgG1, IgG2, IgG3, IgG4, IgA1, and IgA2. The heavy-chain constant domains that correspond to the different classes of immunoglobulins are called α, δ, ε, γ, and μ, respectively. The subunit structures and three-dimensional configurations of different classes of immunoglobulins are well known. “Therapeutic Antibody Engineering” (1^{st} Ed.) Strohl & Strohl Woodhead Publishing (2012)

“Antibody fragments” comprise a portion of an intact antibody, generally the antigen-binding or variable region of the intact antibody. Examples of antibody fragments include Fab, Fab′, F(ab′)2, and Fv fragments; diabodies; single-chain antibody molecules, including single-chain Fv (scFv) molecules; and multispecific antibodies formed from antibody fragments. “Human Monoclonal Antibodies: Methods and Protocols” (2^{nd} Ed.) Steinitz (Ed.) Humana Press (2019).

The term “monoclonal antibody” as used herein refers to an antibody (or antibody fragment) obtained from a population of substantially homogeneous antibodies, e.g., the individual antibodies comprising the population are essentially identical, deriving from a single clone. Monoclonal antibodies are highly specific, being directed against a single antigenic site (epitope). Furthermore, in contrast to polyclonal antibody preparations that typically include different antibodies directed against multiple epitopes, each monoclonal antibody binds a single epitope on the antigen. Monoclonal antibodies are also more convenient to produce than polyclonal antibodies, as monoclonal antibodies are produced in hybridoma cell culture. The modifier “monoclonal” indicates the character of the antibody as being obtained from a substantially homogeneous population of antibodies and is not to be construed as requiring production of the antibody by any particular method. For example, the monoclonal antibodies described herein may be made by the hybridoma method first described by Kohler et al., Nature, 256:495 (1975), or may be made by recombinant DNA methods. See, e.g., U.S. Pat. No. 4,816,567. The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4.816,567; Morrison et al., Proc. Natl. Acad. Sci. USA, 81:6851-6855 (1984)); “Antibody Engineering” Volume 2 (2^{nd} Ed.) Kontermann & Dübel. Springer Press (2010).

A “human antibody” (also called a “fully human antibody”) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

“Humanized” forms of non-human (e.g., murine) antibodies are immunoglobulins, immunoglobulin chains or fragments thereof (such as Fv, Fab, Fab′, F(ab′)2 or other antigen-binding subsequences of antibodies), that contain minimal sequence derived from non-human immunoglobulin. Humanized antibodies can be, for example, human immunoglobulins (recipient antibody) in which residues from a CDR of the recipient are replaced by residues from a CDR of a non-human species (such as mouse, rat or rabbit) or a synthetic sequence (donor antibody), having the desired specificity, affinity, and capacity. Alternatively, an antibody identified or produced in a species other than human, e.g., a mouse antibody, can be “humanized” by substituting one or more amino acids present in the mouse antibody sequence, for the amino acid present in the human antibody sequence at the corresponding sequence position. As there are several sequence differences between, for example, a mouse antibody sequence and a human antibody sequence, substitution for the human position at every site of difference results in a “fully humanized” antibody. In some instances, Fv framework region (FR) residues of the human immunoglobulin are replaced by corresponding non-human residues (“back mutations”). Furthermore, humanized antibodies may comprise residues that are found neither in the recipient antibody nor in the imported CDR or framework sequences. These modifications are made to further refine and optimize antibody performance. In an embodiment, all of the CDRs may be from the donor immunoglobulin in a humanized immunoglobulin. Constant regions need not be present, but if they are, they should be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized or other monoclonal antibodies can have additional conservative amino acid substitutions that have substantially no effect on antigen-binding or other immunoglobulin functions. Humanized immunoglobulins can be constructed by means of genetic engineering. See, e.g., U.S. Pat. No. 5,585,089.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains that enables the scFv to form the desired structure for antigen-binding. For a review of scFv see Pluckthun, in The Pharmacology of Monoclonal Antibodies, vol. 113. Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc Natl Acad Sci USA, 90:6444-6448 (1993).

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. The antibody can be purified, for example, (1) to greater than 95% by weight of antibody as determined by the Lowry method (or optimally, to more than 99% by weight). (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. An isolated antibody can include within its scope, for example, the antibody in situ within recombinant cells since at least one component of the antibody's natural environment is not be present. An isolated antibody is prepared by at least one purification step.

“Epitope,” as used herein, refers broadly to the antigen or portion of an antigen to which an antibody binds. As described herein, the 11-1F4 antibody, for example, binds portions of human light chain amyloid fibrils in an animal, e.g., a mammal, e.g., a human. An epitope having immunogenic activity is a portion of human light chain amyloid fibril that elicits an antibody response, including both binding and effector functions, in an animal. Immunospecificity, the degree to which an antibody binds to a particular epitope, can be determined by methods known in the art, for example, by the immunoassay's described herein. Antigenic epitopes need not necessarily be immunogenic.

### 11-1F4 Antibody-Peptide Epitope Binding Assay

This system and methods described herein provide an 11-1F4 antibody-peptide epitope binding assay with a 10-fold increase in sensitivity compared to similar assays reported in literature. A reliable and highly sensitive binding assay is required during monoclonal antibody drug development processes and for antibody characterization.

The highly sensitive assay described herein provides a reliable quality control assay for 11-1F4 antibody and for any humanized and/or any other modified version of this antibody. The increase in sensitivity compared to previously described assays ensures that differences in affinity of the antibody for its epitope can be more easily observed. The increase in sensitivity also has the advantage of using less reagents in the assay compared to previously described binding ELISAs (Enzyme-Linked Immunoassays).

This peptide ELISA assay uses biotinylated peptide and streptavidin covered plates. This configuration increased the assay sensitivity 10 times (as compared to other peptide ELISA assays).

The use of biotinylated peptide and streptavidin covered plates results in a consistent distribution and conformation of the peptide coating that resulted in a 10-fold increase of assay sensitivity. Higher assay sensitivity of the assay signifies lower possibility for non-specific signal interference, thus providing a better quality control method. This innovation also translates into less antibody required for each individual test thus providing an economic advantage.

The solid supports described herein may be coated with one or both of a LEN peptide comprising the amino acid sequence of DIVMTQSPDSLAVSLGERATIN (SEQ ID NO:1) and/or a control peptide (CT) comprising the amino acid sequence of AVSEHQLLHDKGKSIQDLRRRFFLHHLIAEIHTA (SEQ ID NO:2). A LEN peptide is a peptide derived from the LEN protein, e.g., the 22 amino acid N-terminal fragment (Solomon, A. & Mclaughlin, C., J. Biol. Chem., 244:3303-404, 1969). The LEN peptide and control peptide may be biotinylated, e.g., covalently attached to biotin. Methods for biotinylation of peptides are known in the art.

### Anti-AL Antibodies

11-1F4, also known as CAEL-101, and related variants (Solomon, A, et al., Am. J. Pathol., 137:855-62, 1990; Solomon, A, et al., Clin. Cancer Res., 9:3831s-3838s, 2003; Wall, J. et al., Proc. Natl. Acad. Sci. USA., 115:E10839-E10848, 2018; U.S. Pat. Nos. 8,105,594; 10,046,050; and 10,213,506; U.S. Patent Application Publication No. 2020/0181246), represent an IgG1k monoclonal antibody that binds directly to the conformational epitope present on human light chain amyloid fibrils, regardless of the κ or λ isotype. 11-1F4 has been shown to lead to neutrophil chemotaxis and activation, causing Fcy receptor-mediated opsonization and proteolysis of deposited amyloid fibrils, leading to reversal of AL amyloidomas in mouse models. While not being bound by theory, the 11-1F4 antibody may bind a conformational epitope on human light chain amyloid fibrils. The 11-1F4 antibody may selectively bind to an epitope comprising the amino acid sequence DIVMTQSPDSLAVSLGERATIN (SEQ ID NO:1) (linear epitope). Chimeric, human and humanized (either partially or fully) can be used in the context of this assay.

The immunoassay systems (e.g., enzyme-linked immunoadsorbent assay (ELISA)) and methods described herein may be used to determine the specificity of 11-1F4 antibodies or antigen-binding fragments thereof The binding affinity of antibodies can, for example, be determined by the Scatchard analysis (Frankel, M. & Gerhard, W., Mol. Immunol., 16:101-6, 1979). The binding affinity may be measured, for example, by an antigen/antibody dissociation rate. High binding affinity may be measured by a competition radioimmunoassay. Binding affinity may be measured by ELISA such as the immunoassay described herein.

Immobilization of reagents is required for certain assay methods. Immobilization entails separating the anti-human light chain amyloid fibril antibody from any human light chain amyloid fibril that remains free in solution. This conventionally is accomplished by either insolubilizing the anti-human light chain amyloid fibril antibody before the assay procedure, as by adsorption to a water-insoluble matrix or surface (U.S. Pat. No. 3,720,760), by covalent coupling (for example, using glutaraldehyde cross-linking), or by insolubilizing the anti-human light chain amyloid fibril antibody, e.g., by immunoprecipitation.

### Secondary Antibodies

A detectably-labeled secondary antibody for use with the immunoassay systems and methods described herein may be any antibody or antigen-binding fragment thereof that specifically binds the 11-1F4 antibody or antigen-binding fragment thereof.

The label used with the secondary antibody may be any label with detectable functionality that does not interfere with the binding of human light chain amyloid fibril and anti-human light chain amyloid fibril antibody. Numerous labels are known for use in immunoassays, examples including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamy lase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP. lactoperoxidase, or microperoxidase, biotin/avidin, spin labels, bacteriophage labels, stable free radicals, or combinations thereof. For example, the secondary antibody may be HRP-goat anti-human Fcy.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the antibodies with the above-described fluorescent, chemiluminescent, and enzyme labels (U.S. Pat. No. 3,940,475 (fluorimetry) and U.S. Pat. No. 3,645,090 (enzymes); Hunter et al., Nature. 1962;194:495-496; David et al., Biochemistry. 1974;13:1014-1021; Pain et al., J Immunol Methods, 1981;40:219-230; and Nygren, J Histochem Cytochem. 1982;30(5):407-412). Labels useful for the materials and methods described herein include enzymes such as horseradish peroxidase and alkaline phosphatase.

The conjugation of such label, including the enzymes, to the antibody is a standard manipulative procedure for one of ordinary skill in immunoassay techniques (O'Sullivan et al., “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166).

### Solid Support

The immunoassay system and methods described herein may comprise a solid support. For example, a solid support may be a bead, plate, matrix, polymer, test tube, sheet, culture dish or test strip. A combination of solid supports may be used in the immunoassay systems and methods described herein. The plate may be a multiwell plate. The multiwall plate may be a 2-well plate, 4-well plate, 6-well plate-, 12-well plate, 24-well plate, 48-well plate, 96-well plate, or a 384-well plate. The solid substrate may be constructed of any acceptable material, for example polystyrene, polypropylene, cyclo-olefin, or glass.

The solid support may be coated with the biotinylated LEN peptide comprising the amino acid sequence DIVMTQSPDSLAVSLGERATIN (SEQ ID NO: 1) at a concentration of between about 0.01 μg/mL and 1 μg/mL. For example, the concentration may be about 0.1, 0.15, 0.2, 0.25, 0.3, 0.35, 0.4, 0.45, 0.5, 0.55, 0.65, 0.60, 0.70. 0.75. 0.80, 0.85, 0.90, 0.95, or 1.0 μg/mL. Preferably, the concentration is about 0.1 μg/mL.

The solid support may be coated with the biotinylated control peptide comprising the amino acid sequence AVSEHQLLHDKGKSIQDLRRRFFLHHLIAEIHTA (SEQ ID NO:2) at a concentration of between about 0.01 μg/mL and 1 μg/mL. For example, the concentration may be about 0.1, 0.15, 0.2, 0.25, 0.3, 0.35, 0.4, 0.45, 0.5, 0.55, 0.65, 0.60, 0.70. 0.75, 0.80, 0.85, 0.90, 0.95, or 1.0 μg/mL. Preferably, the concentration is about 0.1 μg/mL.

### Buffers

Buffers may be used in the immunoassay system and methods described herein to form solutions with the 11-1F4 antibodies, wash, block, or a combination thereof. Exemplary buffers include but are not limited to: Coating Buffer comprising Phosphate Buffered Saline (PBS); Wash Buffer comprising PBS and 0.05% TWEEN-20® (Polysorbate 20); Blocking Buffer comprising PBS and 1% BSA (Bovine Serum Albumin); and Assay Buffer comprising PBS, 1% BSA, and 0.05% TWEEN-20® (Polysorbate 20). Buffers are known in the art and commercially available buffers may be used.

### Conditions

Generally, the reagents are incubated for about 60 minutes at about 37° C.

For example, the steps may be for about 30 minutes to 90 minutes. The incubation steps may be for about 60 minutes. For example, the incubation step may be for about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 minutes. The incubation step may be for about 45 to 75 minutes, 55 to 65 minutes, 50 to 70 minutes, or 40 to 80 minutes.

The reaction steps may be performed at about 37° C. For example, the reaction steps may be performed at a temperature between about 35° C, and 45° C. The reaction steps may be performed at a temperature of about 35° C., 36° C., 37° C., 38° C., 39° C., 40° C., 41° C., 42° C., 43° C., 44° C., or 45° C. The reaction steps may be performed at a temperature between about 37° C, and 40° C., 36° C, and 41° C., or 36° C, and 40° C.

### Kits

A kit may comprise one or more containers filled with one or more of the ingredients of the pharmaceutical compositions comprising the buffers, a solid support, optionally a multiwell plate, the LEN peptide (biotinylated), and control peptide(biotinylated). The multiwell plate may be coated with the LEN peptide (biotinylated), and control peptide(biotinylated). Optionally associated with such container(s) can be a notice in the form prescribed by a governmental agency regulating the manufacture, use or sale of pharmaceuticals or biological products, which notice reflects approval by the agency of manufacture, use or sale for human administration.

Kits may further comprise a control antibody that does not react with human light chain amyloid fibril. In another specific embodiment, a kit contains a means for detecting the binding of an antibody to human light chain amyloid fibril (e.g., the antibody may be conjugated to a detectable substrate such as a fluorescent compound, an enzymatic substrate, a radioactive compound or a luminescent compound, or a second antibody that recognizes the first antibody may be conjugated to a detectable substrate). The kit may include a recombinantly produced or chemically synthesized human light chain amyloid fibril. The human light chain amyloid fibril provided in the kit may also be attached to a solid support. In a more specific embodiment, the detecting means of the above-described kit includes a solid support to which human light chain amyloid fibril is attached. Such a kit may also include a non-attached reporter-labeled anti-human antibody. In this embodiment, binding of the antibody to human light chain amyloid fibril can be detected by binding of the said reporter-labeled antibody.

A diagnostic kit for use in screening a biological sample containing antigens of the polypeptide described herein. The diagnostic kit may comprise a substantially isolated antibody specifically immunoreactive with human light chain amyloid fibril, and means for detecting the binding of human light chain amyloid fibril to the antibody. The antibody may be attached to a solid support. The antibody may be a monoclonal antibody. The detecting means of the kit may include a second, labeled monoclonal antibody. Alternatively, or in addition, the detecting means may include a labeled, competing antigen.

In one diagnostic configuration, a biological sample is reacted with a solid phase reagent having a surface-bound human light chain amyloid fibril. After human light chain amyloid fibril binds to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-human light chain amyloid fibril antibody on the solid support. Typically, the reporter is an enzyme that is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, described herein is an assay system or kit for carrying out this diagnostic method. The kit generally includes a support with surface-bound recombinant human light chain amyloid fibril, and a reporter-labeled anti-human antibody for detecting surface-bound anti-human light chain amyloid fibril antibody.

### EXAMPLE 1

### ELISA LEN-c11-1F4 Assay

This example describes the use of the immunoassay system and methods described herein for the c11-1F4 antibody titration against biotinylated peptides (LEN, Control) on a Strepavidin coated plate. Antibody concentration ranged from 5 μg/mL to 5×10⁻⁷ μg/mL, 10× dilution. An IgG1-kappa antibody was used as a negative control.

### Buffers

The following buffers were used: Coating Buffer: PBS (Phosphate Buffered Saline); Wash Buffer: PBS+0.05% TWEEN-20® (Polysorbate 20); Blocking Buffer: PBS+1% BSA (Bovine Serum Albumin); and Assay Buffer: PBS+1% BSA+0.05% TWEEN-20®.

### Plate Coating

50 μL of 0.1 μg/mL PBS solution of biotinylated LEN peptide (DIVMTQSPDSLAVSLGERATIN) (SEQ ID NO:1) (New England Peptide) and 50 μL of 0.1 μg/mL PBS solution of biotinylated Control Peptide (AVSEHQLLHDKGKSIQDLRRRFFLHHLIAEIHTA) (SEQ ID NO:2) were added to each well of a first 12×8 well microplate (StreptaWell Microplate, Transparent, Roche) to make Plate #1.

50 μL of 0.1 μg/mL PBS solution of biotinylated Control Peptide (AVSEHQLLHDKGKSIQDLRRRFFLHHLIAEIHTA) (SEQ ID NO:2) were added to each well of a second 12×8 well microplate (StreptaWell Microplate, Transparent, Roche), to make Plate #2.

The plates were incubated for about 1 hour at about 37° C.

### Blocking

Following incubation for about 1 hour at 37° C., the coated plates were washed three times in a MicroTek plate washer. About 200 μL of blocking buffer was added to each of the wells, and the plates were incubated for about 1 hour at about 37° C.

### Antibody Binding

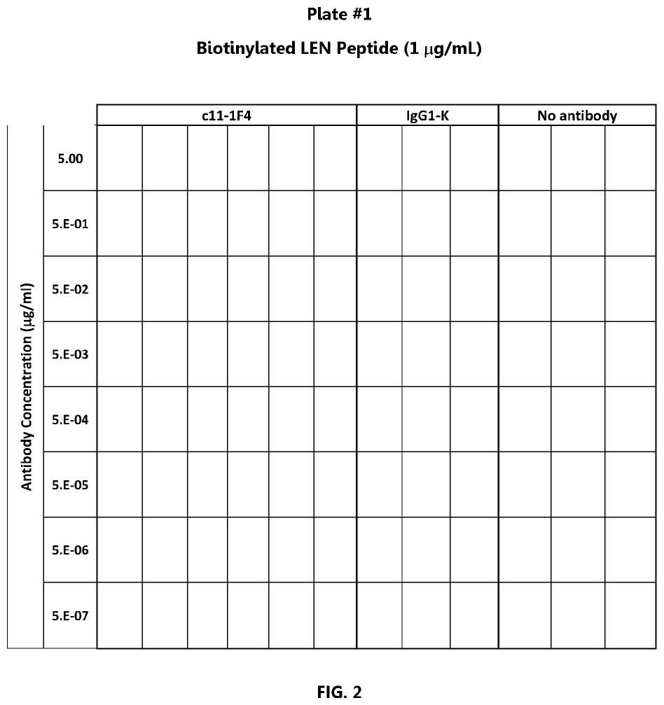

A solution of 5 μg/mL c11-1F4 antibody was prepared in Assay Buffer (c11-1F4 anti-light chain amyloid antibody (TPP-3841) 4.54 mg/mL lot #2753245 from GeneArt). About 140 μL of the 5 μg/mL c11-1F4 antibody solution was aliquoted into the first 6 wells of the first row of an 8×12 well preparatory plate (i.e., Row A, columns 1-6 of the plate shown schematically in FIG. 2).

A solution of 5 μg/mL Human IgG1-K-ULNL was prepared in Assay Buffer (Human IgG1-K-ULNL, Southern Biotech 0151K-01). About 140 μL of the 5 μg/mL Human IgG1-K-ULNL solution was aliquoted into the next three wells in the first row of the preparatory plate (i.e., Row A, columns 7-9 of the plate shown schematically in FIG. 2). The remaining wells in the last three columns (Row A, columns 10-12 of FIG. 2) received 126 μL of Assay buffer. Serial dilutions into the remaining rows (rows B through H of FIG. 2) were performed, starting with 14 μL taken from row A and diluting into 126 μL of buffer in the next row (row B), and then repeating for subsequent rows (i.e., 14 μL from row B into 126 μL of buffer into row C, etc.). From each well of the preparatory plate, 50 μL was transferred to the corresponding wells of each of the two assay plates: Plate #1 and Plate #2 (see, e.g., FIG. 2). The assay plates were incubated for about 1 hour at about 37° C.

### Detection

HRP-goat anti-human Fcy was diluted 1:1000 in assay buffer. The assay plates (Plates #1 and #2) were washed three times in a MicroTek plate washer. 50 μL of a labeled secondary antibody was aliquoted into each well of the assay plates. The assay plates were then incubated for about 1 hour at about 37° C.

### Develop and Read

The assay plates were washed three times in a MicroTek plate washer. 50 μL TMB substrate was aliquoted to each of the wells of the assay plates and the assay plates were incubated at room temperature (about 25° C.) for about 4 minutes. 50 μL of stop solution was aliquoted to each of the wells of the assay plates to halt development. Absorbance was read at 450 nm.

When compared to other ELISA protocols, the method described herein (e.g., ELISA LEN peptide/c11-1F4 antibody) shows an unexpected improvement.

Using an ELISA protocol, O'Nuallain et al. (Biochemistry. 2007;46(45):13049-13058) tested mouse c11-1F4 antibody binding to plate immobilized phage and LEN peptides and the inhibitory effects of these molecules on antibody binding to immobilized Len(1-22), and in Table 1 reported EC₅₀=0.24±0.1 nM.

Using an ELISA protocol, Wall et al. (Proc Natl Acad Sci U S A. 2018;115(46):E10839-E10848) tested chimeric c11-1F4 antibody binding and found that the EC₅₀ (midpoint of the binding transition) was estimated to be ˜0.4 nM, which was indistinguishable from the binding of 11-1F4 to the κ4 N-terminal peptide, LEN (1-22), derived from the Bence Jones protein LEN that served as the immunogen to generate m11-1F4.

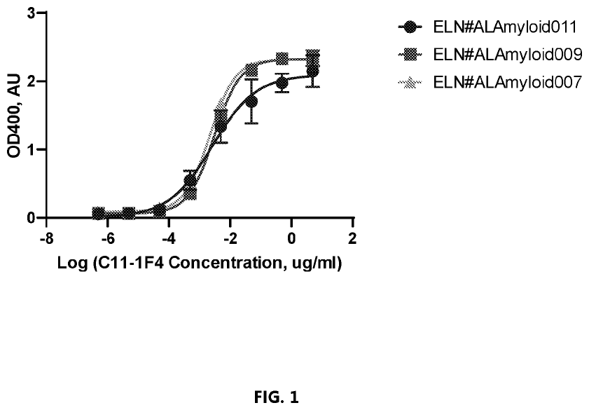

Unexpectedly, the ELISA protocol described herein is over 10 times more sensitive (EC₅₀=0.018±0.001; see FIG. 1) than the ELISA protocol that is routinely used for evaluation of the chimeric c11-1F4 binding to LEN peptide.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. An immunoassay system comprising a solid substrate coated with a biotinylated LEN peptide comprising the amino acid sequence of SEQ ID NO: 1 and a biotinylated control peptide comprising the amino acid sequence of SEQ ID NO:2.

2. The immunoassay system of claim 1, wherein the LEN peptide consists of the amino acid sequence of SEQ ID NO: 1.

3. The immunoassay system of claim 1 or 2, wherein the control peptide consists of the amino acid sequence of SEQ ID NO:2.

4. The immunoassay system of any one of claims 1-3, wherein the solid substrate is coated with the biotinylated LEN peptide at a concentration of between about 0.1 μg/mL and 1 μg/mL.

5. The immunoassay system of any one of claims 1-4, wherein the solid substrate is coated with the biotinylated LEN peptide at a concentration of about 0.1, 0.15, 0.2, 0.25, 0.3, 0.35, 0.4, 0.45, 0.5, 0.55, 0.65, 0.60, 0.70, 0.75, 0.80, 0.85, 0.90, 0.95, or 1.0 1 μg/mL.

6. The immunoassay system of any one of claims 1-5, wherein the solid substrate is coated with the biotinylated LEN peptide at a concentration of about 0.1 μg/mL

7. The immunoassay system of any one of claims 1-6, wherein the solid substrate is coated with the biotinylated control peptide at a concentration of between about 0.1 μg/mL and 1 μg/mL.

8. The immunoassay system of any one of claims 1-7, wherein the solid substrate is coated with the biotinylated control peptide at a concentration of about 0.1, 0.15, 0.2, 0.25, 0.3, 0.35, 0.4, 0.45, 0.5, 0.55, 0.65, 0.60, 0.70. 0.75, 0.80, 0.85, 0.90, 0.95, or 1.0 1 μg/mL.

9. The immunoassay system of any one of claims 1-8, wherein the solid substrate is coated with the control peptide at a concentration of about 0.1 μg/mL.

10. The immunoassay system of any one of claims 1-9, wherein the solid support is a bead, plate, matrix, polymer, test tube, sheet, culture dish or test strip.

11. The immunoassay system of claim 10, wherein the plate is a multiwell plate.

12. The immunoassay system of claim 11, wherein the multiwall plate consists of a 2-well plate, 4-well plate, 6-well plate-, 12-well plate, 24-well plate, 48-well plate, 96-well plate, or a 384-well plate.

13. The immunoassay system of any one of claims 1-12, wherein the solid substrate is a polystyrene, polypropylene, cyclo-olefin, or a glass solid substrate.

14. A kit comprising the immunoassay system of any one of claims 1-13 and a secondary antibody that specifically binds the 11-1F4 antibody or antigen-binding fragment thereof.

15. The kit of claim 14, wherein the antibody is conjugated to a detectable label.

16. The kit of claim 15, wherein the detectable label is a fluorescent label, luminescent label, bioluminescent label, radioactive label, chemiluminescent label, colorimetric label, fluorogenic label, enzymatic label, or a combination thereof.

17. The kit of claim 15 or 16, wherein the label is horseradish peroxidase (HRP).

18. The kit of any one of claims 14-17, wherein the 11-1F4 antibody is a chimeric 11-1F4 antibody (c11-1F4 antibody) or antigen-binding fragment thereof.

19. The kit of any one of claims 14-17, wherein the 11-1F4 antibody is a humanized 11-1F4antibody or antigen-binding fragment thereof.

20. A method for testing a 11-1F4 antibody binding specificity, comprising:
(a) contacting a 11-1F4 antibody or antigen-binding fragment thereof with the immunoassay system of any one of claims 1-13;
(b) washing the solid support;
(c) adding a secondary antibody that specifically binds the 11-1F4 antibody or antigen-binding fragment thereof;
(d) washing the solid support; and
(e) executing detection steps.

21. The method of claim 20, wherein the wash comprises at least 3 washes with a wash buffer.

22. The method of claim 21, wherein the wash buffer comprises PBS and 0.05% TWEEN-20 (polysorbate 20).

23. The method of any one of claims 20-22, wherein the solid support is coated with a biotinylated LEN peptide comprising the amino acid sequence of SEQ ID NO: 1.

24. The method of any one of claims 20-22, wherein the solid support is coated with a biotinylated LEN peptide comprising the amino acid sequence of SEQ ID NO:1, and comprising incubating at solid support with a solution comprising 0.1 μm/mL biotinylated LEN peptide comprising the amino acid sequence of SEQ ID NO: 1 for about 1 hour at about 37° C.

25. The method of claim 24, wherein the solution comprises PBS (Phosphate Buffered Saline).

26. The method of any one of claims 20-25, wherein the solid support is coated with a biotinylated control peptide comprising the amino acid sequence of SEQ ID NO:2.

27. The method of any one of claims 20-26, wherein the solid support is coated with a biotinylated control peptide comprising the amino acid sequence of SEQ ID NO:2, and comprising incubating at solid support with a solution comprising 0.1 μm/mL biotinylated control peptide comprising the amino acid sequence of SEQ ID NO:2 for about 1 hour at about 37° C.

28. The method of claim 27, wherein the solution comprises PBS (Phosphate Buffered Saline).

29. The method of claim 20, wherein the method further comprises a blocking step prior to step (a) comprising incubating the plate with blocking buffer comprising PBS and 1% BSA for about 60 minutes at about 37° C.

30. The method of claim 20, wherein the method further comprises adding a control antibody in the absence of an 11-1F4 antibody to the solid support.

31. The method of claim 30, wherein the control antibody is a human IgG1-k antibody.

32. The method of any one of claims 20-31, wherein the secondary antibody that specifically binds the 11-1F4 antibody or antigen-binding fragment thereof is conjugated to a detectable label.

33. The method of claim 32, wherein the detectable label is a fluorescent label, luminescent label, bioluminescent label, radioactive label, chemiluminescent label, colorimetric label. fluorogenic label, enzymatic label, or a combination thereof.

34. The method of claim 32 or 33, wherein the label is horseradish peroxidase (HRP).

35. A method for testing a 11-1F4 antibody binding specificity comprising
(a) providing a solid support;
(b) coating at least a portion of the solid support with a biotinylated LEN peptide comprising the amino acid sequence of SEQ ID NO: 1 comprising incubating at solid support with a solution comprising 0.1 μm/mL biotinylated LEN peptide comprising the amino acid sequence of SEQ ID NO: 1 for about 1 hour at about 37° C.;
(c) coating at least a portion of the solid support with a biotinylated control comprising the amino acid sequence of SEQ ID NO:2 comprising incubating at solid support with a solution comprising 0.1 μm/mL biotinylated control peptide comprising the amino acid sequence of SEQ ID NO:2 for about 1 hour at about 37° C.;
(d) washing the solid support, optionally at least three times;
(e) contacting the solid support with a blocking buffer comprising PBS (Phosphate Buffer Saline) and 1% BSA (Bovine Serum Albumin) and incubating for about 1 hour at about 37° C.;
(f) contacting a solution comprising a 11-1F4 antibody or antigen-binding fragment thereof with the solid support and incubating for about 1 hour at about 37° C.;
(g) washing the solid support, optionally at least three times;
(h) adding a secondary antibody that specifically binds the 11-1F4 antibody or antigen-binding fragment thereof and incubating for about 1 hour at about 37° C.;
(i) washing the solid support, optionally at least three times; and
(j) executing detection steps.

36. The method of claim 35, wherein the solution comprising a 11-1F4 antibody or antigen-binding fragment thereof comprises assay buffer comprising PBS (Phosphate Buffer Saline), 1% BSA (Bovine Serum Albumin), and 0.05% TWEEN-20 (polysorbate 20).

37. The method of claim 35 or 36, wherein the secondary antibody conjugated to a detectable label.

38. The method of claim 37, wherein the detectable label is a fluorescent label, luminescent label, bioluminescent label, radioactive label, chemiluminescent label, colorimetric label. fluorogenic label, enzymatic label, or a combination thereof.

39. The method of claim 37 or 38, wherein the label is horseradish peroxidase (HRP).

40. The method of any one of claims 35-39, wherein the solution comprising a 11-1F4 antibody or antigen-binding fragment thereof comprises a series of serial dilutions of the solution 11-1F4 antibody or antigen-binding fragment thereof.

41. The method of any one of claims 35-40, wherein the 11-1F4 antibody is a chimeric 11-1F4 antibody (c11-1F4 antibody) or antigen-binding fragment thereof.

42. The method of any one of claims 35-40, wherein the 11-1F4 antibody is a humanized 11-1F4 antibody or antigen-binding fragment thereof.
